(19) United States
(12) Patent Application Publication
(10) Pub. No.: US 2015/0042820 A1
(43) Pub. Date: Feb. 12, 2015

(54) SENSOR SYSTEM FOR DETECTING FUGITIVE GAS

(71) Applicant: Daylight Solutions Inc., San Diego, CA (US)

(72) Inventors: Miles Weida, Poway, CA (US); Timothy Day, Poway, CA (US); William Chapman, San Diego, CA (US)

(21) Appl. No.: 14/452,033

(22) Filed: Aug. 5, 2014

Related U.S. Application Data
(60) Provisional application No. 61/862,655, filed on Aug. 6, 2013.
(60) Provisional application No. 61/909,549, filed on Nov. 27, 2013.

(51) Int. Cl. G01N 21/35; H01S 5/14; G01J 5/00; G01N 21/39; G01N 33/00; H01S 5/34; H01S 5/343

(52) U.S. Cl. CPC G01N 21/3504; H01S 5/3401; H01S 5/14; H01S 5/34313; G01N 21/39; G01N 33/0036; G01J 5/0014; G01N 2021/1765
USPC 348/165

(57) ABSTRACT

A sensor system for sensing the presence of methane and sulfur hexafluoride in an area includes (i) a laser assembly including a quantum cascade gain medium that generates a MIR output beam that is directed at the area; and (ii) an imager that captures a thermal image of the area when the MIR output beam is generated. To sense the presence of methane, the MIR output beam has a center wavelength that is in the range of between approximately 7.654 and 7.668 microns. Alternatively, to sense the presence of sulfur hexafluoride, the MIR output beam has a center wavelength that is in the range of between approximately 10.56 and 10.58 microns.

### CROSS-REFERENCE TO RELATED APPLICATIONS

This application claims priority on U.S. Provisional Application Ser. No. 61/862,655 filed on Aug. 6, 2013 and entitled “SENSOR SYSTEM FOR DETECTING FUGITIVE GAS”, and on U.S. Provisional Application Ser. No. 61/909,549 filed on Nov. 27, 2013 and entitled “SENSOR SYSTEM FOR DETECTING FUGITIVE GAS”. As far as is permitted, the contents of U.S. Provisional Application Ser. No. 61/862,655, and U.S. Provisional Application Ser. No. 61/909,549 are incorporated herein by reference.

### BACKGROUND

Industrial equipment used in the oil, gas, utility, and chemical industries can release gas(es) into the surrounding environment. The escaped gas may be costly, toxic, and/or environmentally harmful. Furthermore, in some applications such as insulating gas used in high-voltage switching equipment, a critical function may be impaired as a result of the gas leak. In many cases, the fugitive gas is odorless, colorless, and may spread quickly. As a result thereof, it can be quite difficult to detect and locate the source of the leak.

### SUMMARY

In one embodiment, the present invention is directed to a sensor system for sensing the presence of methane in an area. In this embodiment, the sensor system can include (i) a laser assembly that generates a first MIR output beam that is directed at the area, the first MIR output beam having a center wavelength that is in the range of between approximately 7.654 and 7.668 microns, the laser assembly including a first quantum cascade gain medium that generates the first MIR output beam; and (ii) an imager that captures a first thermal image of the area when the first MIR output beam is generated.

It should be noted that the acronym “MIR” can be used to describe the term “mid-infrared”. Further the term “MIR range” shall mean and represent the mid-infrared wavelength range of approximately 2-20 micrometers. Further, each output beam can consist of a set of sequential, specific output pulses of light having a center wavelength as specified for each output beam.

In one embodiment, the laser assembly generates a second MIR output beam that is directed at the area, the second MIR output beam having center wavelength that is outside the range of between approximately 7.654 and 7.668 microns but within the mid-infrared range, and the imager captures a second thermal image of the area when the second MIR output beam is generated.

In another embodiment, the sensor system is designed to sense the presence of sulfur hexafluoride in an area. In this embodiment, the sensor system can include (i) a laser assembly that generates a first MIR output beam that is directed at the area, the first MIR output beam having a center wavelength that is in the range of between approximately 10.56 and 10.58 microns, the laser assembly including a first quantum cascade gain medium that generates the first MIR output beam; and (ii) an imager that captures a first thermal image of the area when the first MIR output beam is generated.

In this embodiment, the laser assembly can generate a second MIR output beam that is directed at the area, the second MIR output beam having a center wavelength that is outside the range of between approximately 10.56 and 10.58 but within the mid-infrared range; and the imager can capture a second thermal image of the area when the second MIR output beam is generated.

Moreover, the sensor system can include a control system that blends the first thermal image with the second thermal image to provide a blended image.

Further, the laser assembly can generate a visible output beam that is directed at the area, the visible output beam having center wavelength that is within the visible light spectrum, wherein the imager captures a visible light image of the area when the visible output beam is generated. In this embodiment, the control system can blends the first thermal image, the second thermal image, and the visible light image to provide a blended image.

In one embodiment, the first quantum cascade gain medium directly generates the first MIR output beam without frequency tuning; and the laser assembly includes a second quantum cascade gain medium that generates the second MIR output beam, the second quantum cascade gain medium generating the second MIR output beam without frequency tuning.

In another embodiment, the first quantum cascade gain medium directly generates the first MIR output beam and the second MIR output beam without frequency tuning.

The present invention is also directed to a method for sensing the presence of methane in an area, the method comprising the steps of: directing a first MIR output beam at the area with a laser assembly, the first MIR output beam having a center wavelength that is in the range of between approximately 7.654 and 7.668 microns, the laser assembly including a quantum cascade gain medium that generates the first MIR output beam; and capturing a first thermal image of the area when the first MIR output beam is generated with an imager.

In yet another embodiment, the present invention is directed to a method for sensing the presence of sulfur hexafluoride in an area, the method comprising the steps of: directing a first MIR output beam at the area with a laser assembly, the first MIR output beam having a center wavelength that is in the range of between approximately 10.56 and 10.58 microns, the laser assembly including a quantum cascade gain medium that generates the first MIR output beam; and capturing a first thermal image of the area when the first MIR output beam is generated with an imager.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is simplified illustration of a sensor system having features of the present invention and an emitting gas;

FIG. 2A is graph that illustrates the methane and water spectra near seven and one half (7.5) microns;

FIG. 2B is a graph that illustrates a portion of the methane and water spectra from the graph of FIG. 2A;

FIG. 3 is a graph that illustrates sulfur hexafluoride absorption near ten point six (10.6) microns;

FIG. 4 is a simplified view of a first embodiment of a laser source having features of the present invention;

FIG. 5 is a simplified view of another embodiment of a laser source having features of the present invention;

FIG. 6 is simplified illustration of another embodiment of a sensor system having features of the present invention and an emitting gas;

FIG. 7 is simplified illustration of yet another embodiment of a sensor system having features of the present invention and an emitting gas; and

FIG. 8 is simplified illustration of still another embodiment of a sensor system having features of the present invention and an emitting gas.

### DESCRIPTION

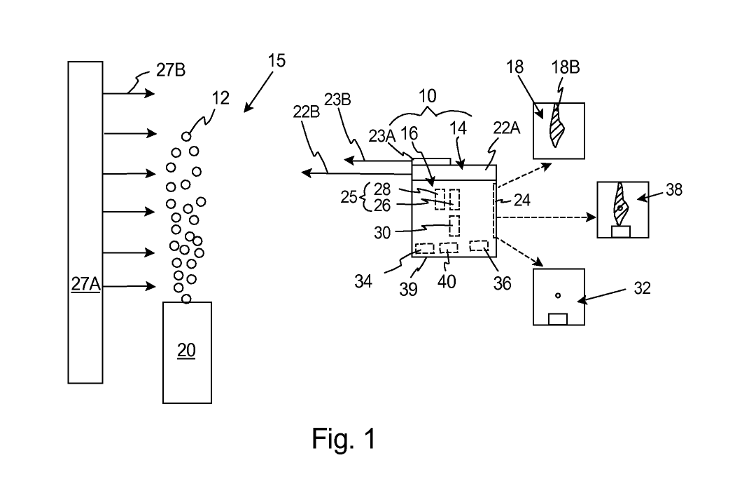

FIG. 1 is simplified illustration of a sensor system 10 having features of the present invention and an emitting, fugitive gas 12 (illustrated as small circles). In this embodiment, the sensor system 10 includes (i) a laser assembly 14 that generates one or more output beams that illuminate an area 15 near the emitting gas 12, and (ii) an imager 16 that captures real-time, high resolution images 18 of the area 15 that can be displayed or recorded for future viewing. As non-exclusive examples, the sensor system 10 is useful for locating emitting gas 12 (i.e. leaks) in the oil, gas, utility, chemical industries. In certain embodiments, because of the unique design disclosed herein, the sensor system 10 is extremely compact, hand-held, lightweight, stable, rugged, small, self-contained, relatively inexpensive, and/or portable.

The type of emitting gas 12 detectable by the sensor system 10 is a gas 12 having molecules that absorb (“absorption features”) in the MIR range. In one embodiment, the sensor system 10 is uniquely designed to sense the presence of sulfur hexafluoride (“SF6”). Alternatively, the sensor system 10 can be uniquely designed to sense the presence of methane (“CH4”). A non-exclusive list of possible gas sources 20 of the emitting gas 12 includes containers, power plants, industrial equipment, gas lines, storage tanks, and electrical equipment.

As an overview, in certain embodiments, the present invention identifies (i) an approximately best center wavelength range (“SF6 wavelength range”) for absorption by sulfur hexafluoride, and (ii) an approximately best center wavelength range (“CH4 wavelength range”) for absorption by methane. Further, the present invention provides (i) a laser assembly 14 that includes a MIR laser source 22A capable of accurately generating an output beam 22B having a center wavelength in the SF6 wavelength range to identify the presence of sulfur hexafluoride gas in the area 15; and (ii) a laser assembly 14 capable of accurately generating an output beam 22B having a center wavelength in the CH4 wavelength range to locate methane gas in the area 15. With this design, a user of the sensor system 10 can direct the output beam 22B at the area 15 to identify the presence of the emitting gas 15.

Additionally, in certain embodiments, the laser assembly 14 can include a visible light source 23A (e.g. a visible light laser) that generates a visible light laser beam 23B (e.g. a red tracer beam) that can be used for providing a reference (aiming) point for the user of the sensor system 10 to determine where the sensor system 10 is pointed to locate a potential leaking of gas 12. With this design, the visible laser beam 23B can be used to approximately identify the area 15 where the output beam 22B is directed.

The imager 16 captures images of the area 15, including the emitting gas 12 and the surrounding environment. In one embodiment, the imager 16 includes an infrared camera 25 that provides real-time, high resolution thermal images 18 (“MIR images”) of the emitting gas 12 that can be displayed on a display 24 and/or recorded for future viewing. In FIG. 1, for convenience, one MIR image 18 from the display 24 is illustrated as being positioned away from the imager 16.

As illustrated in FIG. 1, the output beam 22B can be directed at a background 27A in the area 15, near a possible site of leakage of the gas 12. The infrared camera 25 captures both backscattered light 27B (illustrated as arrows) that is reflected off of the background 27A and background radiation. When a detectable emitting gas 12 is present, the backscattered light 27B is highly attenuated. This produces a region of contrast or shadow 18A in the MIR image 18 that corresponds to and clearly illustrates the emitting gas 12. The background 27A can be any object that reflects the output beams 22B.

In one embodiment, the infrared camera 25 includes a MIR image sensor 26 (illustrated in phantom), and a filter assembly 28 (illustrated in phantom). The MIR image sensor 26 receives the light that passes through the filter assembly 28 and converts the light into electricity. Examples of suitable MIR image sensors 28 can include microbolometer arrays, HgCdTe arrays, quantum well infrared photodetectors (QWIPs), or thermal light valve technology sold by Redshift Systems Corporation, located in Burlington, Mass.

The filter assembly 28 limits the wavelength of the light that is directed at the image sensor 26 to increase contrast in the MIR image 18. For example, in the embodiment for detecting sulfur hexafluoride gas, the filter assembly 28 can be designed to transmit all light approximately in the SF6 wavelength range and block all light having a wavelength that is greater or lesser than the SF6 wavelength range. Alternatively, in the embodiment for detecting methane gas, the filter assembly 28 can be designed to transmit all light approximately in the CH4 wavelength range and block all light having a wavelength that is greater or lesser than the CH4 wavelength range. With this design, the image sensor 26 responds primarily to desired wavelength, and ignores other background thermal emissions.

Alternatively, the filter assembly 28 can be designed to transmit a larger portion of the MIR range including the SF6 wavelength range and the CH4 wavelength range, block all light having a wavelength that is greater or lesser than the larger portion of the MIR range. This will reduce the cost to make the infrared camera 25.

Additionally, the imager 16 can include a visible light camera 30 (illustrated as a box in phantom), a storage system 34 (illustrated as a box in phantom), and a sensor control system 36 (illustrated as a box in phantom). The visible light camera 30 works with light of the visible spectrum and can be used to capture real time, visible light images 32 that can be displayed on the display 24. In FIG. 1, for convenience, one visible light image 32 from the display 24 is illustrated as being positioned away from the imager 16. The visible light image 32 can provide a reference to user of the sensor system 10 to determine where the sensor system 10 is pointed to locate a potential leaking of gas.

In the simplified illustration of FIG. 1, the visible light image 32 includes an image of a circle that represents where the visible light laser beam 23B is pointed and a square that represents an image of the gas source. It should be noted that the visible light laser beam 23B can have other patterns or configurations. For example, the visible light laser beam 23B can form a cross, a spot, or an oval shape. In one embodiment, the visible light laser beam 23B has a circular or oval configuration that approximately matches the spread of the output beam 22B to show the area illuminated by the output beam 22B. In certain embodiments, the visible light laser beam 23B can also be captured with the infrared camera 25.

The storage system 34 stores the various images. Non-exclusive examples of suitable storage systems 34 include flash memory, a floppy disk, a hard disk, or a writeable CD or DVD.

The sensor control system 36 is electrically connected to and controls the operation of the electrical components of the sensor system 10. The sensor control system 36 can include one or more processors and circuits and the sensor control system 36 can be programmed to perform one or more of the functions described herein. The sensor control system 36 (i) receives information from the MIR image sensor 26 and generates the MIR images 18, and (ii) receives information from the visible camera 30 and generates the visible light images 32. Further, the sensor control system 36 can control which of the images 18, 32 is being displayed on the display 24. As non-exclusive examples, the images 18, 32 can be displayed overlaid, side by side at the same time, or the images 18, 32 can be displayed at alternative times.

Additionally, or alternatively, the sensor control system 36 can synthesize (blend) one or more MIR images 18 and one or more visible light images 32 to create one or more synthesized images 38. In FIG. 1, for convenience, one synthesized image 38 from the display 24 is illustrated as being positioned away from the imager 16. In this example, the synthesized image 38 includes the region of contrast or shadow of the emitting gas from the MIR image 18, and the beam and the gas source from the visible image 32. With this design, the IR feature(s) are synthesized with the features from the visible scene.

Additionally, or alternatively, the sensor control system 36 can further enhance the images 18, 38 with color or other features that will further identify the location or type of emitting gas 12. In certain embodiments, the sensor control system 36 can analyze the information from one or more MIR images 18 to identify the specific emitting gas 12 and/or determine the quantity of the emitting gas 12 that is present.

Further, the sensor control system 36 can control the operation of the laser assembly 14. For example, the sensor control system 36 can selectively direct current to each laser source 22A, 22B. The sensor control system 36 can include a sensing circuit that provides feedback relating to the operation of each laser source 22A, 22B, and this feedback can be used for closed loop control of each laser source 22A, 22B.

As provided herein, the sensor control system 36 can direct pulses of power to each laser source 22A, 22B and optimize peak intensity. The duration of each pulse of power and the pulse width (duty cycle) directed by the sensor control system 36 to each laser source 22A, 22B can also be varied. In alternative, non-exclusive embodiments, sensor control system 36 can control each pulse of power to have a duration of approximately 0.010, 0.025, 0.05, 0.075, 0.1, 0.2, 0.3, 0.4, 0.5, 0.6, 0.7, 1, 10, 100, 200, or 500 microseconds. Alternatively, power can continuously be supplied to one or both laser sources 22A, 22B.

In one, non-exclusive embodiment, the sensor control system 36 controls the laser assembly 14 to be pulsed and the refresh rate of the infrared camera 25 to be synchronized with the laser assembly 14. For example, the laser assembly 14 can be controlled to pulse the output beam 22B at the same time as the infrared camera 25 is capturing the images 18. Alternatively, the pulse can be controlled so that the output beam 22B is sometimes on and sometimes off when the infrared camera 25 is capturing the images 18. This will provide a noticeable contrast between these MIR images 18.

In FIG. 1, the imager 16 and the laser source 14 share a common housing 39 and the common sensor control system 36. Alternatively, the imager 16 and the laser source 14 can have separate housings and/or separate control systems.

Additionally, the sensor system 10 can include a battery 40 (illustrated in phantom) for powering the components of the sensor system 10. This allows for the portable operation of the sensor system 10 in remote locations. Alternatively, the sensor system 10 can be connected to another power source, e.g. an electrical cord to a power outlet.

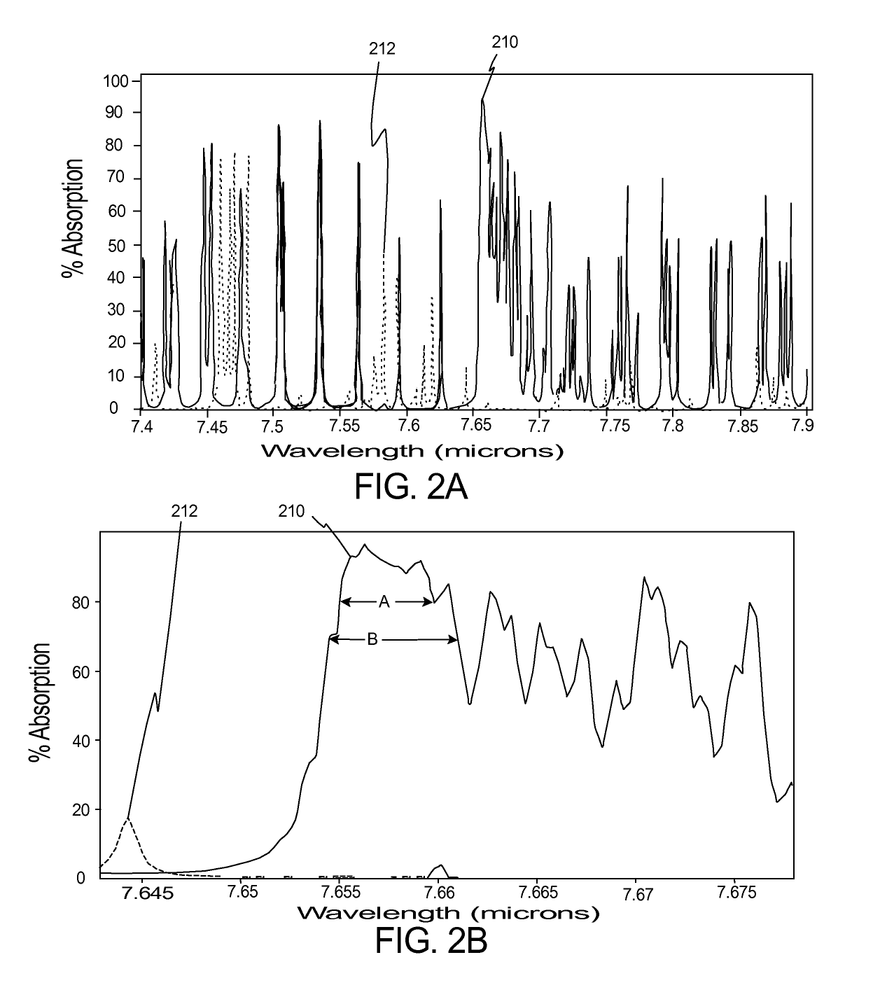

As provided above, the present invention has identified an approximately best center wavelength range (“CH4 wavelength range”) of the output beam 22B for absorption by methane. FIG. 2A is graph that illustrates the methane spectra 210 (illustrated with a solid line) and the water spectra 212 (illustrated with a dashed line) near seven and one-half (7.5) microns.

FIG. 2B is a graph that illustrates a portion of the methane spectra 210 (illustrated with a solid line) and water spectra 212 (illustrated with a dashed line) from the graph of FIG. 2A. More specifically, FIG. 2B illustrates the methane absorption in the range of approximately 7.645 and 7.675 microns. The present invention has identified that near approximately 7.654 microns, the absorption profile of methane is relatively wide and the water absorption is very little. Stated in another fashion, near 7.654 microns, the methane spectra 210 curve is relatively wide. For example, in a first region (designated with A) of the methane spectra 210, the width is approximately 5 nanometers (0.005 microns); and in a second region (designated with B) of the methane spectra 210 the width is approximately 6.5 nanometers (0.065 microns). In one embodiment, the present invention has identified the CH4 wavelength range as a range having a center wavelength of between approximately 7.654 and 7.668 microns. As a result thereof, in one embodiment, the laser assembly 14 provided herein is designed to emit the majority of its optical power in the wavelength range of approximately 7.654 to 7.668 microns.

Because of the relatively wide absorption area of the uniquely chosen CH4 wavelength range, the laser assembly 14 can have relatively loose wavelength tolerances for the output beam 22B, e.g. tolerances of +/−2.5 nanometers. This can be achieved in a small, efficient, and cost effective laser assembly 14.

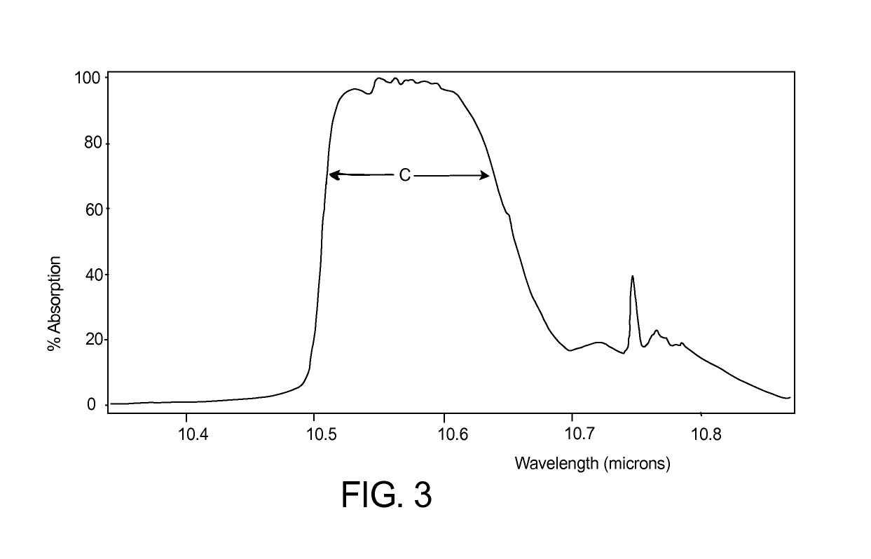

Further, the present invention has identified an approximately best center wavelength range (SF6 wavelength range”) of the output beam 22B for absorption by sulfur hexafluoride. FIG. 3 is graph that illustrates a portion of the SF6 absorption near 10.6 microns. The present invention has identified that near approximately between 10.56 and 10.58 microns, the absorption profile of sulfur hexafluoride is relatively wide. Stated in another fashion, near approximately between 10.56 and 10.58 microns, the curve is relatively wide. For example, in a third region (designated with C), the width is approximately 100 nanometers (0.1 microns). In one embodiment, the present invention has identified the SF6 wavelength range as a range having a center wavelength of between approximately 10.51 and 10.64 microns. As a result thereof, in one embodiment, the laser assembly 14 provided herein is designed to emit the majority of its optical power in the wavelength range of approximately 10.51 and 10.64 microns.

Because of the relatively wide absorption area of the SF6 wavelength range, the laser assembly 14 can have relatively loose wavelength tolerances for the output beam 22B. This can be achieved in a small, efficient, and cost effective Laser assembly 14.

Stated in another fashion, the present invention provides (i) a laser assembly 14 capable of accurately generating the output beam 22B having a center wavelength in the SF6 wavelength range to locate sulfur hexafluoride gas; and/or (ii) a laser assembly 14 capable of accurately generating the output beam 22B having a center wavelength in the CH4 wavelength range to locate methane gas.

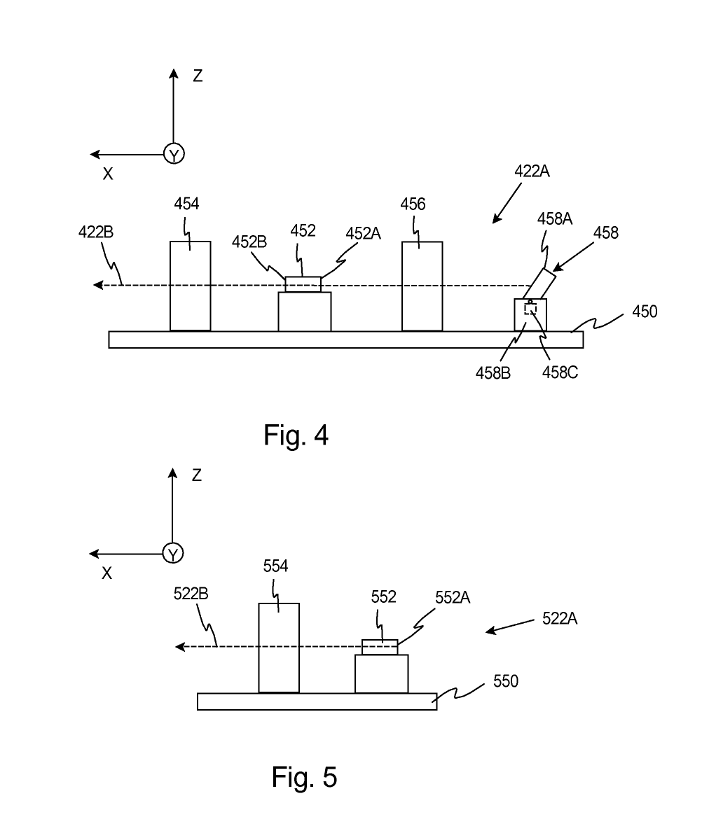

FIG. 4 is a simplified view of a MIR laser source 422A that can used in the sensor system 10 (illustrated in FIG. 1) to accurately generate an output beam 422B that is in the SF6 wavelength range and/or the CH4 wavelength range. In this embodiment, the major components of the laser source 422A include (i) a rigid mounting frame 450, (ii) a gain medium 452, (iii) an output optical assembly 454, (iv) a cavity optical assembly 456, and (v) a wavelength selective (“WS”) feedback assembly 458. The design of each of these components can be varied pursuant to the teachings provided herein. It should be noted that the laser source 422A can be designed with more or fewer components than described above.

In one embodiment, the laser source 422A is an external cavity (EC), narrow linewidth, quantum cascade laser (QCL). With the present design, the output beam 422B can be characterized by near-diffraction limited divergence, narrow linewidth and specific wavelength in the MIR spectral range. Further, the EC-QLC provides stable, predictable spectral emission that does not change over time and does not change due to variations in temperature. In alternative, non-exclusive embodiments, the laser source 422A has a fixed center wavelength of the output beam 422B to within approximately 0.1, 0.01, or 0.001 micrometers.

The mounting frame 450 can be a monolithic structure that retains the gain medium 452, the optical assemblies 454, 456, and the WS feedback assembly 458 in a fixed, stable arrangement to maintain these components in precise mechanical alignment. Further, the mounting frame 450 can be made of a material having a relatively high coefficient to thermal conductivity (e.g. at least approximately 170 watts/meter K) so that heat from the gain medium 452 can be readily transferred. For example, the mounting frame 450 can be fabricated from a single, monolithic structure made of aluminum, copper, copper-tungsten or other material having a sufficiently high thermal conductivity.

Additionally, the laser source 422A can include a temperature adjuster (not shown) that controls the temperature of the gain medium 452 and the temperature of the mounting frame 450. In one non-exclusive embodiment, the temperature adjuster is a thermoelectric cooler (“TEC”) that has approximately the same footprint as the bottom of the mounting frame 450.

In one embodiment, the gain medium 452 is a quantum cascade (“QC”) gain medium. Further, the QC gain medium 452 can use two different semiconductor materials such as InGaAs and AlInAs (grown on an InP or GaSb substrate for example) to form a series of potential wells and barriers for electron transitions. The thickness of these wells/barriers determines the wavelength characteristic of the QC gain medium 452. As used herein, the term QC gain medium 452 shall also include Interband Cascade Lasers (ICL). ICL lasers use a conduction-band to valence-band transition as in the traditional diode laser. In one, non-exclusive embodiment, the semiconductor QCL laser chip is mounted epitaxial growth side down and has a length of approximately four millimeters, a width of approximately one millimeter, and a height of approximately one hundred microns.

In one embodiment, the QC gain medium 452 includes (i) a first facet 452A that faces the cavity optical assembly 456 and the WS feedback assembly 458, and (ii) a second facet 452B that faces the output optical assembly 454. In this embodiment, the QC gain medium 452 emits from both facets. In one embodiment, the first facet 452A is coated with an anti-reflection (“AR”) coating and the second facet 452B is coated with a reflective coating. The AR coating on the first facet 452A allows light directed from the QC gain medium 452 at the first facet 452A to easily exit the QC gain medium 452 and allows the light reflected from the WS feedback assembly 458 to easily enter the QC gain medium 452. In contrast, the reflective coating on the second facet 452B reflects at least some of the light that is directed at the second facet 452B from the QC gain medium 452 back into the QC gain medium 452.

The cavity optical assembly 456 is positioned between the QC gain medium 452 and the WS feedback assembly 458 along the lasing axis (e.g., along the X axis), and collimates and focuses the light that passes between these components. In certain embodiments, the cavity optical assembly 456 can include one lens or more than one lens. For example, in one embodiment, the cavity optical assembly 456 can include an aspherical lens having an optical axis that is aligned with the lasing axis. Moreover, in some embodiments, to achieve the desired small size and portability, the lens has a relatively small diameter. In alternative, non-exclusive embodiments, the lens has a diameter of less than approximately 5 or 10 millimeters, and a focal length of approximately 1 to 20 mm and any fractional values thereof. The lens can comprise materials selected from the group of Ge, ZnSe, ZnS Si, CaF, BaF or chalcogenide glass. However, other materials may also be utilized. The lens may be made using a diamond turning or molding technique. The lens can be designed to have a relatively large numerical aperture (NA). For example, the lens can have a numerical aperture of at least approximately 0.6, 0.7, or 0.8. The NA may be approximated by the lens diameter divided by twice the focal length. Thus, for example, a lens diameter of 5 mm having a NA of 0.8 would have a focal length of approximately 3.1 mm.

The output optical assembly 454 is positioned between the QC gain medium 452 and the window (not shown) in line with the lasing axis. Additionally, the output optical assembly 454 collimates and focuses the light that exits the second facet of the QC gain medium 452. For example, in certain embodiments, the output optical assembly 454 can include one lens or more than one lens that can be somewhat similar in design to the lens or lenses of the cavity optical assembly 456.

The WS feedback assembly 458 reflects light back to the QC gain medium 452 along the lasing axis, and is used to precisely adjust and select the lasing frequency of the external cavity and the wavelength of the output beam 422B. In this manner, the output beam 422B may be tuned and set to a desired fixed wavelength with the WS feedback assembly 458 without adjusting the QC gain medium 452. Thus, in the external cavity arrangements disclosed herein, the WS feedback assembly 458 dictates what wavelength will experience the most gain and thus dominate the wavelength of the output beam 22B.

In certain embodiments, the WS feedback assembly 458 includes a wavelength selective (“WS”) reflector 458A that cooperates with the reflective coating on the second facet 452B of the QC gain medium 452 to form the external cavity. The term external cavity is utilized to designate the WS reflector positioned outside of the QC gain medium 452.

The design of the WS feedback assembly 458 and the WS reflector 458A can vary pursuant to the teachings provided herein. Non-exclusive examples of suitable designs include a diffraction grating, a MEMS grating, prism pairs, a thin film filter stack with a reflector, an acoustic optic modulator, or an electro-optic modulator. Alternatively, a wavelength selective element can be incorporated into the gain medium 452.

In the embodiment illustrated in FIG. 4, the WS feedback assembly 458 can includes a diffraction grating 458A, a grating mount 458B that is secured to the mounting frame 450, and a grating mover 458C that selectively moves the grating 458A to adjust and select the frequency of the output beam 422B. For example, the grating mover 458C can be controlled by the sensor control system 36 (illustrated in FIG. 1).

It should be noted that the laser source 422A can be calibrated using a wavelength measurement device (not shown) during manufacturing. More specifically, with the MIR laser source 422A activated, the WS feedback device 458 can be adjusted until one or more of the desired center wavelength(s) of the output beam 422B is measured with the measurement device. Alternatively, for example, the grating 458A can be manually moved during calibration and subsequently fixedly secured when the desired center wavelength(s) of the output beam 422B is measured with the measurement device.

FIG. 5 is a simplified side view of another embodiment of a laser source 522A that can accurately generate an output beam 522B that is in the SF6 wavelength range or the CH4 wavelength range. In this embodiment, the major components of the laser source 522A includes (i) the mounting frame 550, (ii) the gain medium 552, and (iii) the output optical assembly 554 that are somewhat similar to the corresponding components described above and illustrated in FIG. 4.

However, in this embodiment, the first facet 552A of the gain medium 552 is coated with a highly reflective coating that reflects light back into the gain medium 552. With this embodiment, the design of the gain medium 552 determines the characteristics of the output beam 522B. For example, the gain medium 552 can include a core layer (not shown) that utilizes two different semiconductor materials, such as Indium gallium arsenide (“InGaAs”) and Aluminium indium arsenide (“AlInAs”) to form a series of potential wells and barriers for electron transitions. The wavelength of the output beam 522B of the gain medium 552 can be changed during manufacturing by changing the thicknesses of the periodic layers.

In this embodiment, a plurality of gain mediums 552 can be built and tested, with a gain medium 552 having the desired wavelength characteristics being used for the laser source 514. For example, a gain medium 552 that directly emits an output beam 522B in the SF6 wavelength range will be used for sulfur hexafluoride applications, while a gain medium 552 that directly emits an output beam 522B in the CH4 wavelength range will be used for methane applications.

In either case, the gain medium 552 directly emits the output beam 522B without frequency conversion, tuning, or adjustment. As a result thereof, the laser source 514 is very rugged and not subject to frequency shifts caused by vibration and other disturbances.

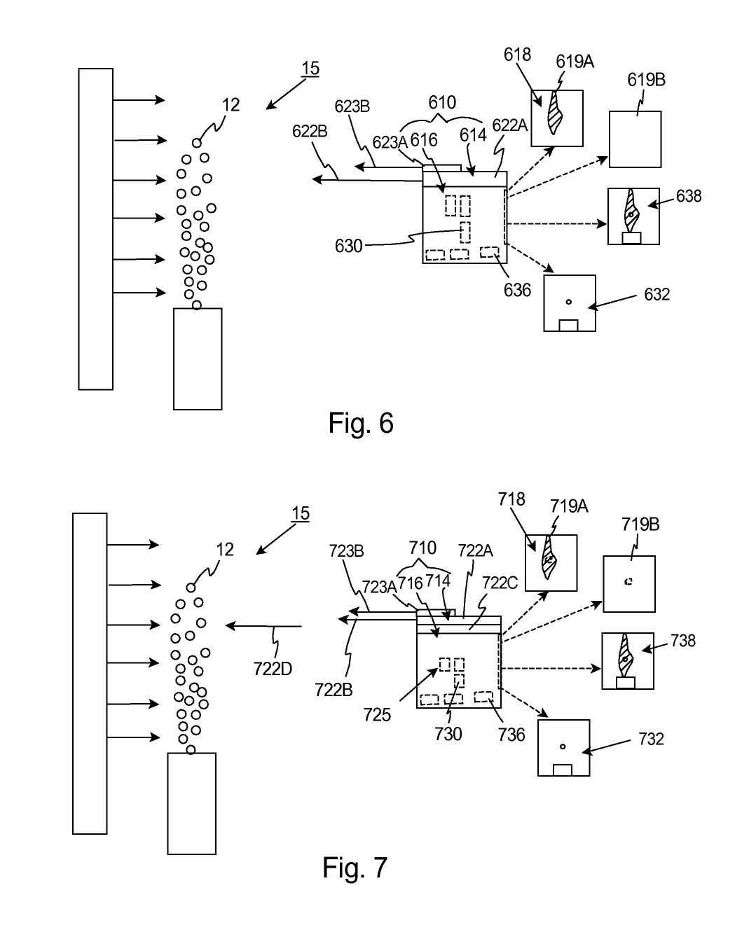

In certain embodiments, it is desired to enhance the contrast of the images captured by the imager. As provided herein, this can be done in a number of ways. For example, FIG. 6 is simplified illustration of another embodiment of a sensor system 610 having features of the present invention and the emitting, fugitive gas 12. In this embodiment, the sensor system 610 includes (i) a laser assembly 614 that illuminates the area 15 near the emitting gas 12, and (ii) an imager 616 that captures real-time, high resolution thermal images 618 of the emitting gas 12 that can be displayed or recorded for future viewing. The components of the sensor system 610 are similar to the components of the sensor system 10 described above and illustrated in FIG. 1.

However, in one embodiment, the sensor control system 636 can control the Mir laser source 622A to be pulsed so that the timing of the pulses are such that the imager 616 captures some images 619A (MIR illuminated images) when the laser light beam 622B is on, and captures some images 619B (non-MIR illuminated images) when the MIR laser light beam 622B is light off. The laser source 622A can be the laser source 422A illustrated in FIG. 4 or the laser source 522A illustrated in FIG. 5. Subsequently, one of the MIR illuminated images 619A can be synthesized with one of the non-MIR illuminated images 619B to create an output image 638 having improved contrast. With this design, the control system 636 can readily identify and the differences between one MIR illuminated image 619A and one subsequently captured non-MIR illuminated image 619B and highlight these differences. In one embodiment, the MIR illuminated image 619A and the non-MIR illuminated image 619B are sequential in time. Alternatively, there can be a time gap between when the MIR illuminated image 619A and the non-MIR illuminated image 619B are captured.

Additionally, in this embodiment, the sensor system 610 can include the visible light laser 623A that generates the visible light laser beam 623B (e.g. a red tracer beam) that can be used for providing a reference (aiming) point for the user of the sensor system 610 to determine where the sensor system 610 is pointed to locate a potential leaking of gas.

Moreover, in this embodiment, the imager 616 can include the visible light camera 630 (illustrated as a box in phantom) that captures light in the visible spectrum. In FIG. 6, for convenience, one visible light image 632 is illustrated as being positioned away from the imager 616. Additionally, or alternatively, the image control system 636 can synthesize the MIR images 618 and the visible light images 632 to create the synthesized image 638.

As provided herein, there are other ways to enhance the contrast of the images provided by the sensor system. For example, FIG. 7 is simplified illustration of another embodiment of a sensor system 710 having features of the present invention and the emitting, fugitive gas 12. In this embodiment, the sensor system 710 includes (i) a laser assembly 714 that includes a first MIR laser source 722A that generates a first MIR beam 722B that illuminates the area 15 near the emitting gas 12, and a second MIR laser source 722C that generates a second MIR beam 722D that illuminates the area 15 near the emitting gas 12, and (ii) an imager 716 that captures real-time, high resolution thermal images 718 of area 15 that can be displayed or recorded for future viewing. In this embodiment, for example, (i) the first MIR laser source 722A is designed so that the first MIR beam 722B is at an absorption window of the gas 12 of interest so that the first MIR beam 722B is at a wavelength that is absorbed if the gas 12 is present, and (ii) the second MIR laser source 722C is designed so that the second MIR beam 722D is at a wavelength that is outside the absorption window of the gas 12 of interest so that the second MIR beam 722B is not absorbed if the gas 12 is present. For example, the laser source 522A illustrated in FIG. 5 can be used for each laser source 722A, 722C. In this embodiment, the gain medium can be designed and selected which directly generate the desired wavelength beam.

In this embodiment, the control system 736 can control the first Mir laser source 722A and the second MIR laser source 722C to be pulsed at different times such that the imager 716 captures some images 719A (first MIR illuminated images) when the first MIR laser light 722B is on, and captures some images 719B (second MIR illuminated images) when the second MIR laser light 722D is light on. Subsequently, one of the first MIR illuminated images 719A can be synthesized with one of the second MIR illuminated images 719B to create an output image 738 having improved contrast. With this design, the control system 736 can readily identify and the differences between one first MIR illuminated images 719A and one subsequently captured second MIR illuminated image 719B and highlight these differences. Further, because both of the images 719A, 719B are illuminated by an MIR beam 722B, 722D, the influence of the beams 722B, 722D on the captured images 719A, 719B can be identified, and this makes it easier to identify if the gas of interest is present. This will allow for a high contrast because there is a big distinction between the fugitive gas and the thermal background created by the beams 722B, 722D.

In this embodiment, the first MIR beam 722B is absorbed by the gas 12 of interest, and the second MIR beam 722D is not. Stated in another fashion, the first MIR beam 722B can be on resonance (having a center wavelength at a peak of the absorption profile of the gas) (e.g. in the SF6 wavelength range or in the CH4 wavelength range), and the second MIR beam 722D can be off resonance (having a center wavelength not in the absorption profile of the gas) (e.g. outside of the SF6 wavelength range or outside of the CH4 wavelength range).

How close the wavelength of the second MIR beam 722D is to the wavelength of the first MIR beam 722B can vary. Generally, the closer the better to make it easier to calibrate. With this design, both images 719A, 719B are captured with almost the same light.

With the present invention, infrared camera 725 can be designed to capture a large wavelength range in the MIR range (broad spectral response), and the laser sources 722A, 722C can be designed to generate very accurate MIR beams 722B, 722D. This way the infrared camera 725 can receive a broad array of thermal information and the accurate beams can be used to illuminate the gas.

Additionally, in this embodiment, the sensor system 710 can include the visible light laser 723A that generates the visible light laser beam 723B, and the imager 716 can include a visible light camera 730 that captures light in the visible spectrum. In FIG. 7, for convenience, one visible light image 732 is illustrated as being positioned away from the imager 716. Additionally, or alternatively, the image control system 736 can synthesize the MIR images 718 and the visible light images 732 to create a synthesized image 738.

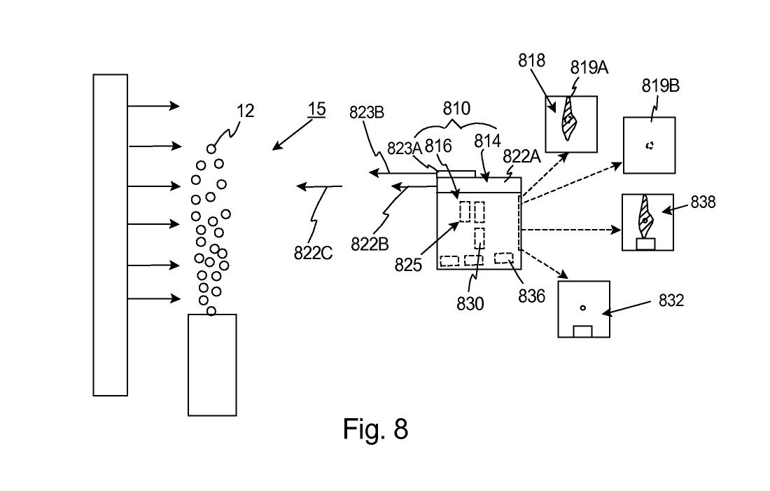

FIG. 8 is simplified illustration of yet another embodiment of a sensor system 810 having features of the present invention and the emitting, fugitive gas 12. In this embodiment, the sensor system 810 includes (i) the laser assembly 814 including a single MIR laser source 822A that generates both a first MIR beam 822B that illuminates the area 15 near the emitting gas 12, and a second MIR beam 822C that illuminates the area 15 near the emitting gas 12, and (iii) an imager 816 that captures real-time, high resolution thermal images 818 of the emitting gas 12 that can be displayed or recorded for future viewing. With this design, a single laser source 822A is used to generate both beams 822B, 822C at different times. In this embodiment, for example, the MIR laser source 822A is designed so that (i) the first MIR beam 822B is at an absorption window of the gas 12 of interest so that the first MIR beam 822B is at a wavelength that is absorbed if the gas 12 is present, and (ii) the second MIR beam 822C is at a wavelength that is outside the absorption window of the gas 12 of interest so that the second MIR beam 822C is not absorbed if the gas 12 is present.

In this embodiment, the control system 836 can control the Mir laser source 822A to pulsed the first MIR beam 822B and the second MIR beam 822C at different times such that the imager 816 captures some images 819A (first MIR illuminated images) when the first MIR laser beam 822B is on, and captures some images 819B (second MIR illuminated images) when the second MIR laser beam 822C is light on. Subsequently, one of the first MIR illuminated images 819A can be synthesized with one of the second MIR illuminated images 819B to create an output image 838 having improved contrast. With this design, the control system 836 can readily identify and the differences between one first MIR illuminated images 819A and one subsequently captured second MIR illuminated image 819B and highlight these differences.

In this embodiment, the first MIR beam 822B is absorbed by the gas 12 of interest, and the second MIR beam 822C is not. How close the wavelength of the second MIR beam 822C is to the wavelength of the first MIR beam 822B can vary. Generally, the closer the better to makes it easier to calibrate.

The design of the MIR laser source 822A can vary. In this embodiment, a single laser chip (e.g. a QC gain medium) can generate both MIR beams 822B, 822C. For example, the laser source 522A of FIG. 5 can be utilized. As provided herein, as the laser source 522A heats up during the pulse, the wavelength of the beam 522B generated by the gain medium 552 will change. With this design, in one embodiment, power is directed to the gain medium 552 in a pulsed fashion so that (i) during a portion (first segment) of the pulse of power, the gain medium 552 generates the second MIR beam 822C; (ii) during another portion (second segment) of the pulse of power (with heating of the chip), the gain medium 552 generates the first MIR beam 822B, and (iii) during yet another portion (third segment) of the pulse of power (with more heating of chip), the gain medium 552 generates a different second MIR beam 822C.

In one embodiment, box car integration can be used to control the pulses of power directed to the gain medium 552 and the capturing of the MIR images 819A, 819B. If have a fast infrared camera, a separate frame could be captured for each portion of the pulse of power, e.g. (i) a frame during the first segment of the pulse (ii) another frame during the second segment of the pulse, and (iii) still another frame during the third segment of the pulse.

Alternatively, if the infrared camera 825 is slower, the camera 825 can take a series of frames during the pulses, and use portion of the frames to capture and combine the frames.

The laser source 822A can be a Fabry perot, a DFB chip, or an external cavity that is churped.

Still alternatively, the laser source 822A can be similar to the laser source 422A illustrated in FIG. 4, in which the grating 458A is fixed, with the heating of the gain medium 452 changing the wavelength. Alternatively, the grating 458A can be selectively movable to change the wavelength of the laser beams 822B, 822C.

Additionally, in this embodiment, the sensor system 810 can include the visible light laser 823A that generates the visible light laser beam 823B, and the imager 816 can include a visible light camera 830 that captures light in the visible spectrum. In FIG. 8, for convenience, one visible light image 832 is illustrated as being positioned away from the imager 816. Additionally, or alternatively, the image control system 836 can synthesize the MIR images 818 and the visible light images 832 to create a synthesized image 838.

It should be noted that a blank frame can always be used randomly (or at a set rate) as a way of background calibration. Still alternatively, the rate of on resonance and off resonance frames can also be randomly set or at a set rate.

While the particular systems as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A sensor system for sensing the presence of methane in an area, the sensor system comprising:
a laser assembly that generates a first MIR output beam that is directed at the area, the first MIR output beam having a center wavelength that is in the range of between approximately 7.654 and 7.668 microns, the laser assembly including a first quantum cascade gain medium that generates the first MIR output beam; and
an imager that captures a first thermal image of the area when the first MIR output beam is generated.

2. The sensor system of claim 1 wherein the laser assembly generates a second MIR output beam that is directed at the area, the second MIR output beam having center wavelength that is outside the range of between approximately 7.654 and 7.668 microns but within the mid-infrared range; wherein the imager captures a second thermal image of the area when the second MIR output beam is generated.

3. The sensor system of claim 2 further comprising a control system that blends the first thermal image with the second thermal image to provide a blended image.

4. The sensor system of claim 2 wherein the laser assembly generates a visible output beam that is directed at the area, the visible output beam having center wavelength that is within the visible light spectrum, wherein the imager captures a visible light image of the area when the visible output beam is generated.

5. The sensor system of claim 4 further comprising a control system that blends the first thermal image, the second thermal image, and the visible light image to provide a blended image.

6. The sensor system of claim 2 wherein the first quantum cascade gain medium directly generates the first MIR output beam without frequency tuning; and wherein the laser assembly includes a second quantum cascade gain medium that generates the second MIR output beam, the second quantum cascade gain medium generating the second MIR output beam without frequency tuning.

7. The sensor system of claim 2 wherein the first quantum cascade gain medium directly generates the first MIR output beam and the second MIR output beam without frequency tuning.

8. A method for sensing the presence of methane in an area, the method comprising the steps of:
directing a first MIR output beam at the area with a laser assembly, the first MIR output beam having a center wavelength that is in the range of between approximately 7.654 and 7.668 microns, the laser assembly including a quantum cascade gain medium that generates the first MIR output beam; and
capturing a first thermal image of the area when the first MIR output beam is generated with an imager.

9. The method of claim 1 further comprising the steps of directing a second MIR output beam at the area with the laser assembly, the second MIR output beam having center wavelength that is outside the range of between approximately 7.654 and 7.668 microns but within the mid-infrared range; and capturing a second thermal image of the area when the second MIR output beam is generated with the imager.

10. The method of claim 9 further comprising the step of blending the first thermal image with the second thermal image to provide a blended image with a control system that includes a processor.

11. A sensor system for sensing the presence of sulfur hexafluoride in an area, the sensor system comprising:
a laser assembly that generates a first MIR output beam that is directed at the area, the first MIR output beam having a center wavelength that is in the range of between approximately 10.56 and 10.58 microns, the laser assembly including a first quantum cascade gain medium that generates the first MIR output beam; and
an imager that captures a first thermal image of the area when the first MIR output beam is generated.

12. The sensor system of claim 11 wherein the laser assembly generates a second MIR output beam that is directed at the area, the second MIR output beam having center wavelength that is outside the range of between approximately 10.56 and 10.58 but within the mid-infrared range; wherein the imager captures a second thermal image of the area when the second MIR output beam is generated.

13. The sensor system of claim 12 further comprising a control system that blends the first thermal image with the second thermal image to provide a blended image.

14. The sensor system of claim 12 wherein the laser assembly generates a visible output beam that is directed at the area, the visible output beam having center wavelength that is within the visible light spectrum, wherein the imager captures a visible light image of the area when the visible output beam is generated.

15. The sensor system of claim 14 further comprising a control system that blends the first thermal image, the second thermal image, and the visible light image to provide a blended image.

16. The sensor system of claim 12 wherein the first quantum cascade gain medium directly generates the first MIR output beam without frequency tuning; and wherein the laser assembly includes a second quantum cascade gain medium that generates the second MIR output beam, the second quantum cascade gain medium generating the second MIR output beam without frequency tuning.

17. The sensor system of claim 12 wherein the first quantum cascade gain medium directly generates the first MIR output beam and the second MIR output beam without frequency tuning.
